SEQUENCE LISTING (4 sequences)

SEQ ID NO: 1 - DNA, 1450 bp, Homo sapiens
  CDS (1)..(1197)
atgcacaccgtggctacgtccggacccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgcgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcgctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgcaccgcctggggtcccacccggcccccgccagggcgcagaagccagggagcagtgggctggccgcgcgcgggctgtgcgtcctgggggaggacaacgcccctctctgagcggacccggtgggaatccgagcggctccctcgggagcggggactgctggaacagcggctattcttctgttattagtatttttcttactgtccaagatcaactgtggaaatattttggtctcttgtgacgttcggtgcagtttcgttgtgaagtttgctattgatattgaaattatgacttctgtgtttcctgaaattaaacatgtgtcaacacaggactttctggatcattccagaaagtgtcagacgtttt

SEQ ID NO: 2 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELHRLGSHPAPARAQKPGSSGLAARGLCVLGEDNAPL

SEQ ID NO: 3 - DNA, 438 bp, Homo sapiens
  CDS (1)..(438)
atgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagccgccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctggggcgcaggtgcggggcagtga

SEQ ID NO: 4 - protein, 145 aa, Homo sapiens [sequence omitted]

(19) United States
(12) Patent Application Publication
(10) Pub. No.: US 2005/0026224 A1
(43) Pub. Date: Feb. 3, 2005

(54) Methods and compositions for modulating G-protein coupled receptor 54

(76) Inventor: Maximillian T. Follettie, Belmont, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/833,829

(22) Filed: Apr. 28, 2004

Related U.S. Application Data
(60) Provisional application No. 60/466,132, filed on Apr. 28, 2003.

(51) Int. Cl. G01N 33/53; G01N 33/567

(52) U.S. Cl. 435/7.2

(57) ABSTRACT

An association is disclosed between the elevated levels of the G-protein coupled receptor 54 (GPR54) and certain cell proliferative diseases, such as kidney cancer. The invention further relates to screening methods to identify compounds that interact with the G-protein coupled receptor to identify agonists and antagonists for diagnosis and treatment. The invention also relates to directed targeting using agents that interact with GPR54, (e.g., anti-GPR54 antibodies or peptide agonists) which are coupled to a toxin.

### REFERENCE TO RELATED APPLICATIONS

This application claims priority to U.S. Provisional Patent Application Ser. No. 60/466,132, filed Apr. 28, 2003, the content of which is expressly incorporated by reference.

### FIELD OF THE INVENTION

The present invention relates to a receptor belonging to the superfamily of G-protein coupled receptors. In particular, the invention pertains to G-protein coupled receptor 54 (GPR54) and its role in solid tissue cancers.

### BACKGROUND OF THE INVENTION

G-protein coupled receptors (GPCRs) constitute a major class of proteins responsible for transducing a signal within a cell. GPCRs have three structural domains: an amino terminal extracellular domain, a transmembrane domain containing seven transmembrane segments, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects intracellular second messengers to extracellular inputs.

GPCR genes and gene-products have been implicated as factors in disease (Spiegel et al., (1993) J. Clin. Invest. 92:1119-1125; McKusick et al., (1993) J. Med. Genet. 30:1-26). These receptors are of critical importance to both the central nervous system and peripheral physiological processes. Evolutionary analyses suggest that the ancestor of these proteins originally developed in concert with complex body plans and nervous systems.

Recently, G-protein coupled receptor 54 (GRP54), also known as hOT7T175 and AXOR12, has been identified as the receptor for the metastin, the product of the Kiss-1 gene (Ohtaki et al., (2001) Nature 411:613-617). Metastin has been shown to suppress cell motility in a chemotaxis assay and wound healing assay (Hori et al., (2001) Biochem Biophys Res Commun 286:958-63). High levels of expression of messenger ribonucleic acid (mRNA) for G-protein coupled receptor 54 have been observed in brain, pituitary gland, and placenta, with the highest levels of KiSS-1 gene expression observed in placenta and brain (Muir et al., (2001) J Biol Chem 276:28969-75). However, only low level expression of mRNA for GRP54 have been found in the normal kidney, and none of the studies have suggested any correlation between GPR54 expression and the presence or growth of solid tumors.

### SUMMARY OF THE INVENTION

It has been discovered that certain cancers are associated with elevated levels of the G-protein coupled receptor 54 (GPR54) receptor. In particular, a link between overexpression of G-protein coupled receptor 54 (GPR54) and kidney cancer is disclosed. The invention further relates to screening methods to identify compounds that interact with the G-protein coupled receptor to identify agonists and antagonists for diagnosis and treatment. The invention also relates to directed targeting using agents that interact with GPR54, (e.g., anti-GPR54 antibodies or peptide agonists) which are coupled to a toxin.

Accordingly, in one aspect, the invention pertains to screening methods for identifying compounds that suppress G-protein coupled receptor-54 (GPR54) expression on cells. Measuring the expression level of the GPR54 receptor on a cell surface in the presence of a test compound, and comparing the measured expression level with a predetermined value whereby useful compounds can be identified when the expression level of the G-protein coupled receptor in the presence of the test compound is lower than the predetermined value (i.e., the level of GPR54 expression in a normal, non-diseased cell such as a normal kidney cell).

The method can also be used to identify agonists of GPR54 by providing a cell that expresses GPR54 on its cell surface, exposing the cell to a candidate agonist, and measuring a signal transduction activity induced by the candidate agonist. The identify of the agonist for GPR54 is based upon measuring the ability of the agonist to induce signal transduction activity. In one embodiment, the step of measuring the signal transduction activity involves measuring levels of at least one protein involved in a G-protein phospholipase C(PLC) pathway. In another embodiment, the step of measuring the signal transduction activity involves measuring the interaction of the GPR54 with at least one arrestin protein.

In such screening assays, the G-protein coupled receptor can be one that is expressed natively in a cell. Alternatively, the G-protein coupled receptor can be a heterologous receptor, and expressed in a cell that has been engineered to express such a receptor. Suitable cells that can be engineered to express the receptor include, but are not limited to, Chinese Hamster Ovary cells (CHO), melanoma cells, HeLa cells, MDCK cells, HEK293 cells, and W138 cells, which are available from the American Type Culture Collection (ATCC, Bethesda Md.). The expression level of the G-protein coupled receptor can be determined by measuring the expression level of a nucleic acid encoding the G-protein coupled receptor, e.g., the mRNA level of GPR54. The expression level of the G-protein coupled receptor can also be determined by measuring the protein level of the G-protein coupled receptor, e.g., GPR54.

In yet another aspect, the invention pertains to a method of inhibiting cell function in a subject expressing aberrant levels of G protein-coupled receptor-54 (GPR54) by exposing the subject to a therapeutically effective amount of a GPR54 binding agent. In one embodiment, the binding agent is a GPR54 agonist, such as metastin or a fragment thereof. In another embodiment, the binding agent is an anti-GPR54 antibody or a binding portion thereof. The binding agent can be used to bind to, and alter the activity of the GPR54.

In yet another embodiment, the binding agent further comprises a toxin, that is toxic to the cells. The toxin can act at the cell surface by binding to, and modifying the GPR54. Alternatively, the toxin can act when it is internalized by the cell, for example, when the toxin or binding agent interacts with the GPR54 and undergoes endocytosis, or binds with at least one arrestin protein, or activates a G-protein phosopholipase C (PLC) pathway, the toxin or binding agent can act to cause an internal effect. Examples of suitable toxins include, but are not limited to, calicheamicin and its derivatives or analogs. In a preferred embodiment, the toxin is calicheamicin.

In yet another aspect, the invention pertains to a method of assessing a cell function or proliferation disease by measuring the expression level of GRP54 in a sample from a subject, and comparing the expression levels of GRP54 with a standard value, whereby a significantly higher expression level is an indicator of a disease state. The cells can be collected for example, from the kidney, or blood. The level of expression can deviate from the normal level and the deviation can thus serve as an indicia of a cell function or proliferation disease, and/or the stage of such disease. For example, the level of GPR54 expression in a normal, non-diseased cell such as a “normal kidney cell” can be about −1.8 ±11.7, determined by fluorescence intensity difference as shown in the Examples section. A diseased state is one that shows a difference from the normal level of expression. For example, a disease state may be indicated when the level of expression of the GPR54 in the test sample differs from the normal level of expression of GPR54 in a subject not afflicted with kidney cancer by a factor of at least about 2, to about 100, preferably by a factor of about 2 to about 90, preferably by a factor of about 2 to about 80, preferably by a factor of about 2 to about 70, preferably by a factor of about 2 to about 60, preferably by a factor of about 2 to about 50, preferably by a factor of about 2 to about 40, preferably by a factor of about 2 to about 30, preferably by a factor of about 2 to about 20, more preferably by a factor of about 2 to about 10, even more preferably by a factor of about 2 to about 5, and most preferably by a factor of about 2 to about 3.

By measuring the difference in expression levels of GPR54, the invention can be used to monitor the progression of kidney cancer in a subject by detecting the presence of GPR54 in a sample from a subject at a first point in time, and at a subsequent point in time; and comparing the expression level of the GPR54, and therefrom monitoring the progression of kidney cancer in the subject.

In addition, by measuring the difference in expression levels of GPR54, the invention can also be used to determine the efficacy of treatment in a subject assessed with kidney cancer by detecting an initial expression level of a G-protein coupled receptor-54 (GPR54) in a sample from a subject. A second expression level of GPR54 at a subsequent point in time occurring after the subject begins treatment is also detected, and then comparing the initial and second level of expression of GPR54 to determine a change in the level of expression as an indicator of efficacy.

In yet another aspect, the invention pertains to a method of inhibiting diseases cell function, e.g., tumor cell function, in a subject with kidney cancer by administering a vector comprising a KiSS-1 gene to kidney cells of the subject, wherein the KiSS-1 gene is operably linked to a promoter functional in kidney cells. The KiSS-1 gene will be expressed to produce metastin, such that the increased amount of metastin in the cell interacts with GPR54 to inhibit cell function. Expression of metastin may results in inhibition of cell function, cell metastasis and tumorogenesis. The KiSS-1 gene can be delivered to the target cells using a vector is selected from the group consisting of adeno-associated virus, lentivirus, adenovirus, rectrovirus.

In yet another aspect, the invention pertains to a method of specifically targeting kidney cancer cell expressing aberrant levels of G protein-coupled receptor-54 (GPR54) exposing the kidney cancer cell to a therapeutically effective amount of a GPR54 binding agent; and measuring the expression level of the GPR54 receptor, wherein a reduction in the expression level of the GPR54 receptor indicates that the cell has been specifically targeted.

The binding agent can be a GPR54 agonist, such as the GPR54 ligand, metastin or a fragment thereof. Alternatively, the binding agent can be an anti-GPR54 antibody. The binding agent can further comprise a toxin that can be selectively delivered to cells that express aberrant levels of GPR54. The toxin can be calicheamicin, or other toxins selected from the group consisting of disulphide analogs of dihydro derivatives of calicheamicin, N-acylated derivatives of calicheamicin, and esperamicins. The toxin can act at the cell surface by binding to, and modifying the GPR54. Alternatively, the toxin can act when it is internalized by the cell, for example, when the toxin or binding agent interacts with the GPR54 and undergoes endocytosis, or binds with at least one arrestin protein (cellular protein involved in endocytosis), or activates a G-protein phosopholipase C (PLC) pathway, the toxin or binding agent can act to cause an internal effect.

### BRIEF DESCRIPTION OF FIGURES

FIG. 1 is a bar graph showing the average GPR54 mRNA expression levels from normal kidney cells versus clear kidney cancer cells; and

FIG. 2 is a bar graph showing the individual GPR54 mRNA expression levels from normal kidney cells versus clear kidney cancer cells;

FIG. 3 is a vector showing the insertion site for GPR54.

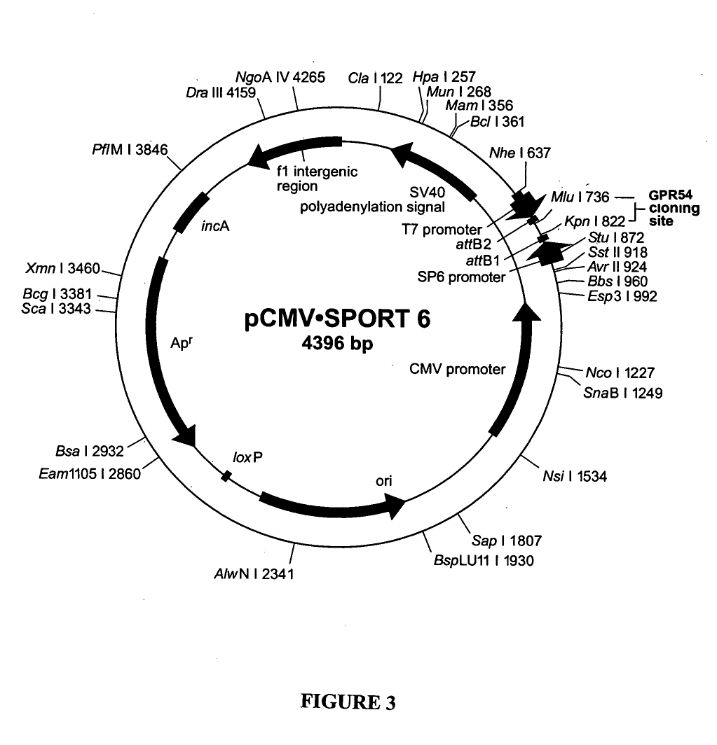

### DETAILED DESCRIPTION

The practice of the present invention employs, unless otherwise indicated, conventional methods of microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.))

So that the invention may more readily be understood, certain terms are first defined:

The term “GPR54” or “G protein coupled receptor 54” or “AXOR12” or “hOT7T175” refer to a G-protein coupled receptor (Genbank Accession No. AJ309020, and Entrez AB051065). The nucleotide sequence of human GPR54 is shown in SEQ ID NO: 1, and the amino acid sequence is shown in SEQ ID NO: 2. GPR54 is a 398 amino acid protein that contains seven hydrophobic transmembrane domains (See e.g., Muir et al., (2001) J. Biol. Chem. 276: 28969-28975; and Kotani et al., (2001) J. Biol. Chem. 276: 34631-34636). GPR54 has sequence homology with class I members of the GPCR. However, GPR54 exhibits high sequence homology (81% amino acid identity) to rat GPR54 (Lee et. al. (1999) FEBS LETT 446:103-107).

The term “metastin” as used herein refers to the peptide ligand agonist for GPR54. Metastin is the protein product of the Kiss-1 gene (Genbank Accession No. A4029541). The nucleotide sequence of human metastin is shown in SEQ ID NO: 3, and the amino acid sequence is shown in SEQ ID NO: 4. The Kiss-1 is a human metastasis suppressor gene (See Lee et al., (1997) J Natl Cancer Inst, 88:1731-1737). The Kiss-1 gene has been shown to suppress metastasis without affecting tumorogenicity in human melanomas (Lee et al., (1997) Int. J. Cancer 71:1035-1044) and breast carcinomas (Lee et al., (1997) Cancer Res. 57:2384-2387). The Kiss-1 gene encodes a 145 amino acids protein, named “metastin.” Metastin inhibits chemotaxis and invasion of GPR54 transfected CHO cells in vitro and attenuates pulmonary metastasis in GPR54-transfected B16-BL6 melanomas in vivo (See Ohtaki et al., (2001) Nature 411:613-617). Biologically active fragments of metastin that are smaller than the entire ligand are

Also within the scope of the invention are peptides and homologous peptides that function with a similar activity as metastin. The peptides can be any size less than the full length polypeptide as long as they retain biological activity. For example, the peptides can be about 40 amino acids to about 3 amino acids in length, preferably about 20 amino acids to about 3 amino acids, more preferably about 15, 14, 13, 12, 11, 10, 9, 8, 7, 6, 5, 4 and 3 amino acids in length. Preferably, the peptides are 54 amino acids in length (amino acids 68-121 of SEQ ID NO: 4), the peptides are 14 amino acids in length (amino acids 108-121 of SEQ ID NO: 4), and the peptides are 13 amino acids in length (amino acids 109-121 of SEQ ID NO: 4). The peptides can be synthesized using standard peptide synthesis techniques known in the art.

The term “homology” or “identity” or “homologous” as used herein refers to the percentage of likeness between nucleic acid molecules. To determine the homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identify” is equivalent to amino acid or nucleic acid “homology”). The percent identify between the two sequences is a function of the number of identical positions shared by the sequences, taking into account the number of gaps, and the length of each gap, which need to be introduced for optimal alignment of the two sequences.

The comparison of sequences and determination of percent identify between two sequences can be accomplished using a mathematical algorithm. For example, the percent identity between two amino acid sequences can be determined using the Needleman and Wunsch (1970) J. Mol. Biol. (48):444-453) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, and 6. In another example, the percent identify between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In yet another example, the percent identify between two amino acid or nucleotide sequences is determined using the algorithm of E. Meyers and W. Miller (CABIOS, 4:11-17(1989)) which has been incorporated into the ALIGN program (version 2.0), using a PAM120 weight residue table, a gap length penalty of 12.

The term “toxin” as used herein refers to a substance that destroys a cell. The destruction of the cell, for example, via a biological process such as apoptosis. Alternatively, the destruction of the cell may be at the level of a nucleic acid, for example, a toxin that introduces breaks in DNA strands. A preferred toxin is calicheamicin (Mylotarg®; CMA-676, Wyeth Pharmaceuticals, Philadelphia, Pa.), or analogs or derivatives thereof. The calicheamicin can be “linked” or “coupled” to a binding agent (e.g., a ligand agonist for GPR54 or an anti-GPR54 antibody) such that an interaction between the binding agent and the cell expressing elevated levels of GPR54, results in targeted delivery of the toxin to the cell. Calicheamicin causes double strand breaks in DNA and has been used in antibody-targeted chemotherapy in patients with acute myeloid leukemia (AML) (See e.g., Larson et al., (2002) Leukemia 16:1627-1636; Voutsadakis (2002) Anticancer Drugs 13:685-692; Hamann et al., (2002) Biocongugate Chem 13:40-46). Details of the calicheamicin toxin can be found in U.S. Pat. No. 4,970,198, and details of calicheamicin related toxins can be found in U.S. Pat. Nos. 4,675,187: 4,539,203: 4,554,162; and 4,837,206. Details of calicheamicin derivatives can be found in U.S. Pat. Nos. 5,037,651 and 5,079,233, and of calicheamicin-antibody conjugates in U.S. Pat. No. 5,773,001.

The term “binding agent” refers to a compound that interacts with GPR54. Examples of binding agents include, but are not limited to, anti-GPR54 antibodies, or fragments thereof, ligand agonists such as metastin or peptides thereof and synthetic molecules that bind to GPR54.

The term “operably linked” as used herein refers to an arrangement of elements wherein the components are configured so as to perform their usual function. Thus, control elements operably linked to a coding sequence are capable of effecting the expression of the coding sequence, so long as they function to direct the expression of the coding sequence. For example, intervening untranslated yet transcribed can be present between a promoter sequence and the coding sequence and the promoter sequence can still be considered “operably linked” to the coding sequence.

The phrase “inhibit cell function” as used herein refers slowing down, reducing or stopping cell propagation, or cell proliferation, or cell growth and division, communication, movement, cell turnover cell production, cell creation, and cell movement.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. As used herein the term “antibody” also includes functional fragments of antibodies.

The term “antigen-binding portion” of an antibody or an “antibody portion” includes fragments of an antibody that retain the ability to specifically bind to an antigen (e.g., GPR54 receptor). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (See e.g., Holliger et al., (1993) Proc. Natl. Acad. Sci. USA 90:6444-6448; Poljak, et al., (1994) Structure 2:1121-1123).

Still further, an antibody or antigen-binding portion thereof may be part of larger immunoadhesion molecules, formed by covalent or non-covalent association of the antibody or antibody portion with one or more other proteins or peptides. Examples of such immunoadhesion molecules include use of the streptavidin core region to make a tetrameric scFv molecule (Kipriyanov, et al. (1995) Human Antibodies and Hybridomas 6:93-101) and use of a cysteine residue, a marker peptide and a C-terminal polyhistidine tag to make bivalent and biotinylated scFv molecules (Kipriyanov et al., (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

An “isolated antibody” includes an antibody that is substantially free of other antibodies having different antigenic specificities (e.g., an isolated antibody that specifically binds GPR54 is substantially free of antibodies that specifically bind antigens other than GPR54). Moreover, an isolated antibody may be substantially free of other cellular material and/or chemicals.

The term “modulate” or “modify” are used interchangeably herein and refer to the up-regulation or down-regulation of a target gene or a target protein. The term modifies or modified also refers to the increase, decrease, elevation, or depression of processes or signal transduction cascades involving a target gene or a target protein. A target protein can be a receptor, for example, a G protein coupled receptor, e.g., GPR54. Modification of the GPCR receptor may occur when an antibody to the GPCR binds to the GPCR. In a preferred embodiment, the GPCR is GPR54. The modification may directly affect the GPR54 receptor, for example modifications that result in decrease in GPR54 receptor number expression. Alternatively, the modifications may occur as an indirect effect of binding to the target protein. For example, binding of a binding agent such as a ligand agonist or an anti-GPR54 antibody that leads to a change in downstream processes involving the GPR54, such as activation of signal transduction pathways that reduce the GPR54 receptor available for binding, for example by endocytosis. The modifications can therefore be direct modifications of the target protein, or an indirect modification of a process or cascade involving the target protein. Non-limiting examples of modifications includes modifications of morphological and functional processes, under-or over production or expression of proteins that, e.g., inhibit cell proliferation, cell activity, cell migration, chemotaxis and cell tumorogenicity.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The phrase “compounds that suppress” as used herein refers to agents that reduce in the expression level of a G protein coupled receptor by a defectable amount. In particular, the phrase refers to compounds that reduce the expression level of GPR54 from an aberrantly high level to a normal level.

The phrase “signaling pathway” as used herein refers to the modulation (e.g., stimulation or inhibition) of a cellular function/activity upon the binding of a binding agent, e.g., an agonist or antibody, to the GPCR (e.g., GPR54). Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase; polarization of the plasma membrane; production or secretion of molecules; alteration in the structure of a cellular component; cell function, e.g., synthesis of DNA; cell migration; cell differentiation, cell proliferation; and cell survival.

The terms “agonist” and “antagonist” represent compounds that enhance or diminish a response. As one form of an agonist, the compound binds to the same site as the endogenous compound and produces the same type of signal, usually of equal or greater magnitude than the endogenous agent. Another form of agonist binds to a different site than the first agonist, producing no signal by itself, however, an enhanced signal is generated when the endogenous agent also binds to its site. This is called an allosteric action. One form of antagonist binds to the site used by the endogenous agent and diminishes or blocks the signal generated by the endogenous agent. Another form of antagonist binds to an allosteric site, similar to the second form of agonist, but produces a diminished signal generated by the endogenous agent. A third form of antagonist dissolves in the membrane or crosses the membrane and intercepts the signal generated by the endogenous agent within the membrane or on the intracellular side. An antagonist, accordingly, encompasses negative agonists or “inverse agonists”, having a negative intrinsic activity that reduces the receptor signal activity relative to the signaling activity measured in the absence of the inverse agonist. Such an antagonist is distinguished from an antagonist having no intrinsic activity and no effect on the receptor's basal activity. (See, Milligan et al., TIPS 16:10 (1995).) The invention is described in more detail in the following sections:

I. G-protein Coupled Receptors and Signal Transduction Pathways

The G-protein coupled receptor-54 (GPR54) receptor protein is a GPCR that participates in signaling pathways. These pathways refers to the modulation (e.g., stimulation or inhibition) of cellular function/activity upon the interaction of a binding agent (e.g., with the GPCR (e.g., GPR54)). Examples of signal transduction pathways include those involved in mobilization of molecules such as phosphatidylinositol 4,5-bisphosphate (PIP₂), inositol 1,4,5-triphosphate (IP₃) and adenylate cyclase; polarization of the plasma membrane; production or secretion of molecules; alteration in the structure of a cellular component; cell function, e.g., cell proliferation, synthesis of DNA; cell migration; cell differentiation; and cell survival.

The response mediated by the receptor protein depends on the type of cell. For example, in some cells, binding of the binding agent to the receptor protein can stimulate an activity such as release of compounds, gating of a channel, cellular adhesion, migration, differentiation, etc., through phosphatidylinositol or cyclic AMP metabolism and turnover while in other cells, the binding of the binding agent can produce a different result. Regardless of the cellular activity/response being modulated, the receptor is a GPCR and upon interaction with the binding agent, the receptor interacts with G proteins to produce one or more secondary signals, in a variety of intracellular signal transduction pathways, e.g., through phosphatidylinositol or cyclic AMP metabolism and turnover.

Phosphatidylinositol turnover or cyclic AMP metabolism involves molecules involved in the turnover and metabolism of phosphatidylinositol 4,5-bisphosphate (PIP₂). PIP₂ is a phospholipid found in the cytosolic leaflet of the plasma membrane. Binding of a binding agent, in particular, an agonist ligand, to the receptor activates, in some cells, the plasma-membrane enzyme phospholipase C which in turn can hydrolyze PIP₂ to produce 1,2-diacylglycerol (DAG) and inositol 1,4,5-triphosphate (IP₃). Once formed IP₃ can then diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, refers to an activity of PIP₂ or one of its metabolites.

Another signaling pathway in which the receptor may participate is cyclic AMP (cAMP) pathway which involves the turnover and metabolism of cAMP as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

II. G-proteins

G proteins represent a family of heterotrimeric proteins composed of alpha (α), beta (β), and gamma (γ) subunits, that bind guanine nucleotides. These proteins are usually linked to the cell surface GPCR's. Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cAMP (e.g., by activation of adenyl cyclase), diacylglycerol or inositol phosphates. Greater than 20 different types of α-subunits are known in humans. These subunits associate with a smaller pool of β and γ subunits. Examples of mammalian G proteins include Gi, Go, Gq, Gs and Gt. G proteins are described extensively in Lodish et al., Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al., eds., Academic Press (1994).

III. GPR Ligands

In one aspect, the invention pertains to using agonists or ligands of GPR54 coupled to a toxin as a method for directed targeting for cells that overexpress GPR54. In one embodiment, metastin is the preferred agonists, and can be used by linking the agonist to a toxin, such as calichaemian and its derivatives or analogs. Peptides of metastin are also within the scope of the invention.

These peptides should have sufficient similarity/homology so as to perform one or more of the same functions as performed by metastin. Similarity is determined by conserved amino acid substitution. Such conservative substitutions are those that substitute a given amino acid in a polypeptide by another amino acid of like characteristics. Conservative amino acid substitutions can be made at one or more amino acid residues. A “conservative amino acid substitution” in one in which the amino acid residue is replaced with an amino acid residue having a similar side chain. Families of amino acid residues having similar side chains have been defined in the art, including basic side chains (e.g. lysine, arginine, histidine), acidic side chains (e.g., aspartic acid, glutamic acid), uncharged polar side chains (e.g., glycine, asparagines, glutamine, serine, threonine, tyrosine, cysteine), nonpolar side chains (e.g., alanine, valine, leucine, isoleucine, proline, phenylalnine, methionine, tryptophan), beta-branched side chains (e.g., threonine, valine, isoleucine), and aromatic side chains (e.g., tyrosine, phenylalanine, tryptophan, histidne). Guidance concerning which amino acid changes that are conservative can be found in Bowie et al., (1990) Science 247:1306-1310 and Stryer, Biochemistry, Third Ed., 1988.

A variant peptide is also encompassed with the scope of the inventions and is one which can differ in amino acid sequence by one or more substitutions, deletions, insertions, inversions, fusions, and truncations or a combination of any of these. Variant polypeptides can be fully functional or can lack function in one or more activities. Thus, in the present case, variations can affect the function, for example, of one or more of the regions corresponding to ligand binding, membrane association, G-protein binding and signal transduction.

Functional variants typically contain only conservative substitutions or variations in non-critical residues or in non-critical regions. Functional variants can also contain substitution of similar amino acids which result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

Variants can be naturally-occurring or can be made by recombinant means or chemical synthesis to provide useful and novel characteristics for the receptor polypeptide. This includes preventing immunogenicity from pharmaceutical formulations by preventing protein aggregation.

Useful modifications further include alteration of ligand binding characteristics. For example, one embodiment involves a modifications at the binding site that results in binding but not release, or slower release, of ligand. A further useful modifications at the same sites can result in a higher affinity for ligand. Useful modifications also include changes that provide for affinity for another ligand. Another useful modifications includes one that allows binding but which prevents activation by the ligand. Another useful modifications includes variation in the transmembrane G-protein-binding/signal transduction domain that provides for reduced or increased binding by the appropriate G-protein or for binding by a different G-protein than the one with which the receptor is normally associated. Another useful modifications provides a fusion protein in which one or more domains or subregions is operationally fused to one or more domains or subregions from another G-protein coupled receptor.

Amino acids that are essential for function can be identified by methods known in the art, such as site-directed mutagenesis or alanine-scanning mutagenesis (Cunningham et al., (1989) Science 244:1081-1085). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., (1992) J. Mol. Biol. 224:899-904; de Vos et al., (1992) Science 255:306-312).

IV. Production of Recombinant Proteins

In one embodiment, the proteins (e.g., GPR54, anti-GPR antibodies, agonist ligands, etc.) are produced by recombinant DNA techniques. For example, a nucleic acid molecule encoding the receptor polypeptide is cloned into an expression vector, the expression vector introduced into a host cell and the receptor protein expressed in the host cell. The receptor protein can then be isolated from the cells by an appropriate purification scheme using standard protein purification techniques.

Polypeptides can contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., (1990) Meth. Enzymol. 182: 626-646) and Rattan et al., (1992) Ann. N.Y. Acad. Sci. 663:48-62.

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications. The same type of modification may be present in the same or varying degree at several sites in a given polypeptide. Also, a given polypeptide may contain more than one type of modification.

V. Production of Antibodies

Antibodies to the antigens for direct transfer of immunity can also be administered. Antibodies can be generated using standard techniques known in the art and include recombinant antibodies, chimeric antibodies, humanized antibodies, and the like. The antibody may be of animal, e.g., a mouse or rat. Preferably, the antibody is a human antibody. The antibody may be a chimeric antibody (See e.g., Morrison et al., (1984) Proc Nat. Acad. Sci. U.S.A. 81: 6851-6855) or a humanized antibody (See e.g., Jones et al., (1986) Nature 321: 522-525. Methods of producing antibodies suitable for use in the present invention are well known to those skilled in the art and can be found described in such publications as Harlow & Lane, Antibodies: A Laboratory Manual, Cold Spring Harbor Laboratory (1988), and Asai, Methods in Cell Biology Vol. 37. Antibodies in Cell Biology, Academic Press, Inc. N.Y. (1993).

When antibodies are generated by immunizing animals with an antigen to yield antibody which is not of human origin, the antibodies could elicit an adverse effect if administered to humans. Methods for “humanizing” antibodies, or generating less immunogenic fragments of non-human antibodies, are well known. A humanized antibody is one in which only the antigen-recognized sites, or complementarity-determining hypervariable regions (CDRs) are of non-human origin, whereas all framework regions (FR) of variable domains are products of human genes. These “humanized” antibodies present a lesser xenografic rejection stimulus when introduced to a human recipient.

To accomplish humanization of a selected mouse monoclonal antibody, the CDR grafting method described by Daugherty et al. (1991) Nucl. Acids Res. 19:2471-2476, may be used. Briefly, the variable region DNA of a selected animal recombinant anti-idiotypic ScFv is sequenced by the method of Clackson, et al., (1991), Nature, 352:624-688. Using this sequence, animal CDRs are distinguished from animal framework regions (FR) based on locations of the CDRs in known sequences of animal variable genes (See e.g., Kabat, et al., (1991) Sequences of Proteins of Immunological Interest, Fifth Edition, U.S. Department of Health and Human Services, NIH Publication No. 91-3242). Once the animal CDRs and FR are identified, the CDRs are grafted onto human heavy chain variable region framework by the use of synthetic oligonucleotides and polymerase chain reaction (PCR) recombination. Codons for the animal heavy chain CDRs, as well as the available human heavy chain variable region framework, are built in four (each 100 bases long) oligonucleotides. Using PCR, a grafted DNA sequence of 400 bases is formed that encodes for the recombinant animal CDR/human heavy chain FR protection.

For recombinant production of antibodies, the sequences of human heavy chain constant region genes are known in the art (See e.g., Kabat, et al. Supra) and DNA fragments encompassing these regions can be obtained by standard PCR amplification. The heavy chain constant region can be an IgG1, IgG2, IgG3, IgG4, IgA, IgE, IgM or IgD constant region and any allotypic variant therein as described in Kabat et al., supra The sequences of human light chain constant region genes are known in the art and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (See e.g., Bird et al., (1988) Science 242:423-426; Huston et al., (1988) Proc. Natl. Acad. Sci. USA 85:5879-5883; McCafferty et al., (1990) Nature 348:552-554).

To express the antibodies, or antibody portions, DNAs encoding partial or full-length light and heavy chains are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Additionally or alternatively, the recombinant expression vector can encode a signal peptide that facilitates secretion of the antibody chain from a host cell.

The recombinant expression vectors used for antibody production carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; (1990) Gene Expression Technology. Methods in Enzymology 185, Academic Press, San Diego, Calif. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma.

For expression of the light and heavy chains, the expression vector(s) encoding the heavy and light chains can be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in Kaufman et al., (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. In addition, bifunctional antibodies may be produced in which one heavy and one light chain are an antibody of the invention and the other heavy and light chain are specific for an antigen other than GPR54 by crosslinking an antibody of the invention to a second antibody by standard chemical crosslinking methods. The structure of several G-protein coupled receptors is available from known GPCR databases. Multiple alignments of various G-protein coupled receptor families are also available at known sites.

Due to the high degree of homology in the 7-transmembrane domains of the GPCR receptors, identification of novel members of this superfamily, as well identification of the extracellular portions of such novel members, is readily accomplished by those of skill in the art. By way of example, the book of Watson and Arkinstall (1994), incorporated herein by reference, provides the sequences of over 50 members of the 7-transmembrane domain G-protein-coupled receptor superfamily. The book further describes, for each sequence, the precise residues comprising the transmembrane domains. The “extracellular portions” of these receptors are the regions between the N-termini and the start of the first transmembrane domains.

Sequence comparison between the different GPCRs revealed that all these receptors have in common a central core domain consisting of seven transmembrane helices (TM-I through-VII) connected by three intracellular (i 1, i2 and i3) and three extracellular (e1, e2 and e3) loops (Baldwin (1993) EMBO J., 12, 1693-1703). Two cysteine residues (one in e1 and one in e2) which are conserved in most GPCRs, form a disulfide link which is probably important for the packing and for the stabilization of a restricted number of conformations of these seven TMs.

Particularly important amino acids of GPR54 are amino acids 40-62, 76-99, 117-140, 158-181, 189-212, 244-268 and 277-300 which are considered to constitute the transmembrane segments of the GPR54 receptor. The DNA sequences coding for the transmembrane segments are considered to be highly homologous with other G-protein coupled receptor coding fragments.

Amino acids that constitute the extracellular loops of the GPR54 receptor will be of particular importance as targets for antibodies intended for clinical use in e.g. targeted drug delivery or for drug design. Amino acids that constitute the intracellular loops of the GPR54 receptor will be particularly important in the elucidation of the mechanisms for the coupling of the GPR54 receptor to G-proteins. These regions may serve as targets for drugs aiming for the modulation of the interaction of the receptor with G-proteins. Such altered receptors may be desired to further the understanding of the molecular mechanisms in the coupling of the receptor with G-proteins.

VI. Screening Assays

The receptor polypeptides e.g., GRP54 (including variants, fragments and analogs thereof) are useful for biological assays related to GPCRs. Such assays involve any of the GPR54 functions or activities or properties useful for diagnosis and treatment of GPR54 related conditions. Diseased tissue, such as kidney cancer tissue, can be used to identify modified receptor levels which then serve as a basis for diagnosis and for rational drug design.

The receptor polypeptides are also useful in drug screening assays, in cell-based or cell-free systems. Cell-based systems can be native, i.e., cells that normally express the receptor protein, as a biopsy or expanded in cell culture. In one embodiment, however, cell-based assays involve recombinant host cells expressing the receptor protein, e.g., GPR54. Diseased tissue, and specifically diseased kidney tissue, is useful to screen for drugs that can be used in affecting the diseased tissue. Preferred cells include, but are not limited to, kidney cells and blood cells. The effect of the drug can be scored based on, among other things, morphological change.

The polypeptides can be used to identify compounds that modulate receptor activity. The GPR54 receptor protein cancer can be used in high-throughput screens to assay candidate compounds for the ability to bind to the receptor and ameliorate a disease such as cancer. The cancer can be ameliorated by inhibiting cell function, proliferation of cells, inhibiting cell chemotaxis or cell metastasis. These compounds can be further screened against a functional receptor to determine the effect of the compound on the receptor activity. Compounds can be identified that activate (agonist) or inactivate (antagonist) the receptor to a desired degree.

The receptor polypeptides can be used to screen a compound for the ability to stimulate or inhibit interaction between the receptor protein and a target molecule that normally interacts with the receptor protein (e.g., the interaction of GPR54 with its ligand agonist, metastin.) The target can be ligand or a component of the signal pathway with which the receptor protein normally interacts (for example, a G-protein or other interactor involved in cAMP or phosphatidylinositol turnover and/or adenylate cyclase, or phospholipase C activation). The assay includes the steps of combining the receptor protein with a candidate compound under conditions that allow the receptor protein or fragment to interact with the target molecule, and to detect the formation of a complex between the protein and the target or to detect the biochemical consequence of the interaction with the receptor protein and the target, such as any of the associated effects of signal transduction, such as ion flux, G-protein phosphorylation, cyclic AMP or phosphatidylinositol turnover, and adenylate cyclase or phospholipase C activation.

The receptor polypeptides, e.g., GPR54, are useful in cell based assays when they are overexpressed in a cell. Accordingly, such cells overexpressing the receptor are useful to identify compounds that are capable of modulating or compensating for the overexpression. Cells overexpressing the receptor can be derived from natural sources or can be created by routine recombinant methods.

The receptor polypeptides, e.g., GPR54, are also useful for screening compounds in a cell based assay when constitutively activated on a cell. Such cells expressing constitutively activated receptors are useful for screening compounds that modulate receptor activation. Such cells can be derived from natural sources or can be created by recombinant means that are well known in the art. (See e.g., Scheer et al., (1997) J. Receptor Signal Transduction Res. 17:57-73; U.S. Pat. No. 5,750,353).

Candidate compounds include, for example, 1) peptides such as soluble peptides, including Ig-tailed fusion peptides and members of random peptide libraries (See, e.g., Lam et al., (1991) Nature 354:82-84; Houghten et al., (1991) Nature 354:84-86) and combinatorial chemistry-derived molecular libraries made of D- and/or L-configuration amino acids; 2) phosphopeptides (e.g., members of random and partially degenerate, directed phosphopeptide libraries, (See, e.g., Songyang et al., (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble full-length receptor or fragment that competes for ligand binding. Other candidate compounds include mutant receptors or appropriate fragments containing mutations that affect receptor function and thus compete for ligand. Accordingly, a fragment that competes for ligand, for example with a higher affinity, or a fragment that binds ligand but does not allow release, is encompassed by the invention.

The invention provides other end points to identify compounds that modulate (stimulate or inhibit) receptor activity. The assays typically involve an assay of events in the signal transduction pathway that indicate receptor activity. Thus, the expression of genes that are up- or down-regulated in response to the receptor protein dependent signal cascade can be assayed. In one embodiment, the regulatory region of such genes can be operably linked to a marker that is easily detectable, such as luciferase. Alternatively, phosphorylation of the receptor protein, or a receptor protein target, could also be measured.

Any of the biological or biochemical functions mediated by the receptor can be used as an endpoint assay. These include all of the biochemical or biochemical/biological events described herein, in the references cited herein, incorporated by reference for these endpoint assay targets, and other functions known to those of ordinary skill in the art.

The receptor polypeptides, e.g., GPR54, are also useful in competition binding assays in methods designed to discover compounds that interact with the receptor. Thus, a compound is exposed to a receptor polypeptide under conditions that allow the compound to bind or to otherwise interact with the polypeptide. Soluble receptor polypeptide is also added to the mixture. If the test compound interacts with the soluble receptor polypeptide, it decreases the amount of complex formed or activity from the receptor target. This type of assay is particularly useful in cases in which compounds are sought that interact with specific regions of the receptor. Thus, the soluble polypeptide that competes with the target receptor region is designed to contain peptide sequences corresponding to the region of interest.

To perform cell-free drug screening assays, it is desirable to immobilize either the receptor protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/GPR54 fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule; as well as enzyme-linked assays which rely on detecting an enzymatic activity associated with the target molecule.

Modulators of receptor protein activity identified according to these drug screening assays can be used to treat a subject with a disorder mediated by the receptor pathway, by treating cells that express the GPR54 protein in tubular organs such as the kidney and ovaries. These methods of treatment include the steps of administering the modulators of protein activity in a pharmaceutical composition as described herein, to a subject in need of such treatment.

The receptor polypeptides also are useful to provide a target for diagnosing a disease, or predisposition to disease mediated by the GPR54 protein, especially in the kidney. Preferred cells include, but are not limited to, kidney cells, ovary cells, endometrial cells, and blood cells. Accordingly, methods are provided for detecting the presence, or levels of, the receptor protein in a cell, tissue, organ, or organism. The method involves contacting a biological sample with a compound capable of interacting with the receptor protein such that the interaction can be detected.

One agent for detecting receptor protein is an antibody capable of selectively binding to receptor protein, e.g., an anti-GPR54 antibody that binds with GPR54. A biological sample includes tissues, cells and biological fluids isolated from a subject, as well as tissues, cells and fluids present within a subject.

The receptor protein also provides a target for diagnosing active disease, or predisposition to disease, in a patient having a variant receptor protein. Thus, receptor protein can be isolated from a biological sample, assayed for the presence of a genetic mutation that results in aberrant receptor protein. This includes amino acid substitution, deletion, insertion, rearrangement, (as the result of aberrant splicing events), and inappropriate post-translational modification. Analytic methods include altered electrophoretic mobility, altered tryptic peptide digest, altered receptor activity in cell-based or cell-free assay, alteration in ligand or antibody-binding pattern, altered isoelectric point, direct amino acid sequencing, and any other of the known assay techniques useful for detecting mutations in a protein.

In vitro techniques for detection of receptor protein include enzyme linked immunosorbent assays (ELISAs), Western blots, immunoprecipitations and immunofluorescence. Alternatively, the protein can be detected in vivo in a subject by introducing into the subject a labeled anti-receptor antibody. For example, the antibody can be labeled with a radioactive marker whose presence and location in a subject can be detected by standard imaging techniques. Particularly useful are methods which detect the allelic variant of a receptor protein expressed in a subject and methods which detect fragments of a receptor protein in a sample.

The receptor polypeptides are also useful in pharmacogenomic analysis. Pharmacogenomics deal with clinically significant hereditary variations in the response to drugs due to altered drug disposition and abnormal action in affected persons. See, e.g., Eichelbaum, (1996) Clin. Exp. Pharmacol. Physiol. 23:983-985, and Linder (1997) Clin. Chem. 43:254-266. The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes effects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants of the receptor protein in which one or more of the receptor functions in one population is different from those in another population. The polypeptides thus allow a target to ascertain a genetic predisposition that can affect treatment modality. Thus, in a ligand-based treatment, polymorphism may give rise to amino terminal extracellular domains and/or other ligand-binding regions that are more or less active in ligand binding, and receptor activation. Accordingly, ligand dosage would necessarily be modified to maximize the therapeutic effect within a given population containing a polymorphism. As an alternative to genotyping, specific polymorphic polypeptides could be identified.

The receptor polypeptides are also useful for monitoring therapeutic effects during clinical trials and other treatment. Thus, the therapeutic effectiveness of an agent that is designed to increase or decrease gene expression, protein levels or receptor activity can be monitored over the course of treatment using the receptor polypeptides as an end-point target.

The receptor polypeptides are also useful for treating a receptor-associated disorder. Accordingly, methods for treatment include the use of soluble receptor or fragments of the receptor protein that compete for ligand binding. These receptors or fragments can have a higher affinity for the ligand so as to provide effective competition.

VII. Toxins

In one aspect, the invention pertains to directed toxin targeting by coupling or linking a cell toxin to a binding agent that interacts with GPCR, e.g., GPR54. This allows specific delivery of the toxin molecule to cells that express elevated levels of GPR54. In a preferred embodiment, the toxin is calicheamicin (“LL-E33288 complex”) (U.S. Pat. Nos. 4,970,198; 5,053,394; 4,671,958; 4,970,198; 5,037,651, and 5,079,233).

Calicheamicin is a hydrophobic enediyne antibiotic that binds noncovalently to DNA and causes sequence-selective oxidation of deoxyribose. Calicheamicin causes double-stranded breaks, and cells exposed to it eventually become apoptotic. The drug makes several base contacts along the minor groove, however, the diversity of binding-site sequences and the effects of DNA conformation on calicheamicin-induced DNA cleavage suggest that sequence recognition per se is not the primary determinant of target selection. Calicheamicin interaction typically involves DNA bending as part of an induced-fit mechanism of DNA target recognition (Salzberg et al. (2000) Biochemistry 39:7605-12).

Calicheamicin has been used for acute myeloid leukemia (AML) (Wyeth Pharmaceuticals, Philidelphia, Pa.). Gemtuzumab ozogamicin is a novel monoclonal antibody-targeted chemotherapy agent for AML. Gemtuzumab ozogamicin is a humanized recombinant anti-CD33 monoclonal antibody linked to calicheamicin, a potent cytotoxic agent. The antibody targets the CD33 antigen found in elevated levels on the surface of leukemic blast cells and myeloid precursors. After ligation with the CD33 on the cell surface, gemtuzumab ozogamiun is internalized and hydrolyzed. Its two components are released into the cytoplasm and calicheamicin enters the nucleus where it associates with the DNA, causing double helix breaks and finally cell death. This targeting effect reduces the toxicity of gemtuzumab ozogamicin. The efficacy and tolerability of gemtuzumab ozogamicin have been documented in AML (Shannon-Dorcy K (2002) Oncol Nurs Forum 29:E52-9; Hamann et al., (2002) Bioconjug Chem 13:47-58).

In one embodiment the binding agent is an antibody or antibody portion. Linkers used to generate antibody-calicheamicin conjugates have been described (See, e.g., Hamann et al., (2002) Bioconjug Chem 13:40-46) Previous calicheamicin conjugates relied on the attachment of a hydrazide derivative to the oxidized carbohydrates that occur naturally on antibodies. This results in a “carbohydrate conjugate” capable of releasing active drug by hydrolysis of a hydrazone bond in the lysozomes where the pH is low. In one embodiment, the antibody can be linked to the calicheamicin by the attachment of a hydrazide derivative to an oxidized carbohydrate on the antibody, e.g., an anti-G protein coupled receptor antibody, e.g., an anti-GPR54 antibody.

Conjugates have also been made that are formed by reacting a calicheamicin derivative containing an activated ester with the lysines of antibodies. This results in an “amide conjugate” that is stable to hydrolysis, leaving the disulfide that is present in all calicheamicin conjugates as the likely site of drug release from the conjugate. In another embodiment, the antibody can be linked to the calicheamicin by reacting a calicheamicin derivative containing an activated ester with a lysine amino acid residue of the antibody, e.g., an anti-G protein coupled receptor antibody, e.g., an anti-GPR54 antibody.

Using the same methodology the anti-GPR54 antibody can be coupled to calicheamicin and used to target cells that express elevated levels of GPR54. The anti-GPR54 antibody can be obtained commercially, for example from Pheonix Pharmaceuticals, Inc Belmont, Calif. Alternatively, the anti-GPR54 antibody or an antigen binding portion can be prepared using standard known techniques for antibody production.

The toxins can be linked to the antibody using the linking techniques described in U.S. Pat. Nos. 5,773,001, 5,767,285, 5,767,285, 5,739,116, 5,714,586, and 5,712,374 (herein incorporated by reference). The antibodies can be conjugated to the toxin using linkers. The linkers require a carbonyl group on one end for formation of a Schiff's base, particularly a hydrazone, and a carboxylic acid on the other end. The carboxylic acid can be activated and subsequently reacted with the lysines of an antibody or other targeting protein or with an amine, alcohol, or other appropriate nucleophile on other targeting agents which have been chosen for their ability to target undesired cell populations (See U.S. Pat. No. 5,773,001). Antibody carriers can be from almost any mammalian species (eg. mouse, human, dog, etc.) and can be produced by various methods (e.g. murine antibodies via hybridomas, human antibodies via hybridomas from transgenic mice, etc). Preferably, the antibody is a humanized antibody or antibody fragment. The basic technology for humanization is disclosed by Winter in U.S. Pat. No. 5,225,539 and by Adair in WO 91/09967. The antibody can be attached to the toxin using the methods.

These antibody-toxin conjugates can be produced when the two protein components are covalently linked. Generally, the components are chemically coupled. However, the linkage may also be a peptide or disulfide bond. The antibody directs the toxin to cell types presenting a specific antigen thereby providing a specificity of action not possible with the natural toxin.

Also within the scope of the invention are toxins derivatives or variants of calicheamicin that can be used to specifically target cells expressing GPR54. The toxins derivatives or variants include, but are not limited to, disulphide analogs of calicheamicin (U.S. Pat. No. 5,773,001), dihydro derivatives (U.S. Pat. No. 5,037,651), and N-acylated derivatives (U.S. Pat. No. 5,079,233). Related compounds include, but are not limited to, esperamicins (U.S. Pat. Nos. 4,675,187; 4,539,203; 4,554,162; and 4,837,206). All of these compounds contain a methyltrisulfide that can be reacted with appropriate thiols to form disulfides, at the same time introducing a functional group such as a hydrazide or similar nucleophile. Examples of this reaction with the calicheamicins are given in U.S. Pat. No. 5,053,394 which also discloses targeted forms of the calicheamicins. All information in the above-mentioned patent citations is incorporated herein by reference.

In preferred embodiment, the binding agent is an agonist of the GPR. In this embodiment, the invention pertains to directed delivery of calicheamicins using a ligand agonist of GPR54, such as metastin or peptides thereof. The calicheamicins can be linked to the peptides by peptide-link forming steps that may be any of those conventionally used in peptide synthesis. For instance the p-nitrophenyl ester method described initially by Bodansky may be used (See Bodansky (1955) Nature 175, 685), or, the N,N′-dicyclohexylcarbodiimide method described by Sheehan et al (See Sheehan et al (1955) J. Am. Chem. Soc. 77, 1067). Preferably, the peptides are linked to the toxin by C-terminal or N-terminal coupling. Using linkers is also within the scope of the invention, e.g., non-polynucleotide linkers that are described in, among other places, in several volumes of the series Methods in Enzymology, Academic Press, San Diego Calif.

In a preferred embodiment, metastin or peptide fragments thereof can be covalently attached to N-acetyl-gamma calicheamicin dimethyl hydrazide via bifunctional AcBut (4-[4′-acetylphenoxyl] butanoic acid linker. The ligand-toxin reagent can be used to demonstrate selective killing of cancer derived cells expressing GPR54 protein relative to vector-alone transfected controls. Cell death can be monitored by standard proliferation assays including measuring incorporation of radiolabeled nucleotides into DNA as function of time or by colorimetric assay of reduction of the tetrazolium salt MTT as assay of mitochondrial function. Specificity of drug delivery via GPR54 of the ligand peptide-toxin construct can be shown by demonstration of the requirement for GPR54 expression in target cell and metastin-derived sequence specificity in ligand-toxin conjugate for maximal drug-induced toxicity. Similarly, specificity of targeting can be demonstrated by competition with metastin sequence derived peptide ligand(s) alone that block specific uptake of ligand-toxin conjugates.

Alternatively, metastin or peptide fragments thereof can be synthesized with a covalently attached amino-terminal fluorescein isothiocyanate (FITC) tag using standard peptide synthesis. Internalization of metastin or derived peptide ligands can be demonstrated by internalization of fluorescent-tagged peptide ligand(s) for GPR54. GPR54 receptor mediated endocytosis can be monitored by fluorescence microscopy and demonstrated by internalization of FITC-ligand in GPR54 expressing cell lines, using vector-alone transfected cell lines as control.

Also within the scope of the invention, is the identification of downstream genes resulting from metastin/GPR54 activation. These genes can be assessed by detecting the alteration of gene expression profile of the downstream genes mediated by stimulation or interaction of GPR54 with ligand (e.g., metastin). The attainment of metastatic potential by transformed cells is a critical step in the morbidity of cancer. Signal transduction mediated by GPR54 stimulated with metastin or derived peptide ligands has been demonstrated to decrease the metastatic potential of melanoma cell lines and the migration of tranfected CHO cells. The downsteam genes associated with cancer cell metastasis can be identified by the alteration of gene expression profile mediated by stimulation GPR54 with ligand. Using cancer cell lines transfected with the GPR54 gene expression construct, the cellular mRNA concentration can be profiled using Affymetrix oligonucleotide arrays following stimulation with metastin derived peptide ligands and compared to unstimulated controls. The cell lines that can be used for this analysis include, but are not limited to, the melanoma-derived LOX cell line as well as breast (MDA-435) or colon (SW480, HT29, LoVo) cell lines (Available from American Type Culture Collection (Rockville, Md.)). The ligand/GPR54 mediated gene expression will be measured from cells grown either in-vitro or in mouse xenograft models. The mRNA expression altered by ligand stimulation of GPR54 will identify key genes in the metastatic phenotype of cancers and identify novel targets for chemotherapeutic intervention.

VIII. Vectors/Host Cells

The invention also provides vectors containing the receptor polynucleotides, e.g., GRP54. The term “vector” refers to a vehicle, preferably a nucleic acid molecule, that can transport the receptor polynucleotides. When the vector is a nucleic acid molecule, the receptor polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC. The vectors can be used to express the receptor polynucleotides for assays, such as screening assays.

A vector can be maintained in the host cell as an extrachromosomal element where it replicates and produces additional copies of the receptor polynucleotides. Alternatively, the vector may integrate into the host cell genome and produce additional copies of the receptor polynucleotides when the host cell replicates.

The invention provides vectors for the maintenance (cloning vectors) or vectors for expression (expression vectors) of the receptor polynucleotides. The vectors can function in prokaryotic or eukaryotic cells or in both (shuttle vectors).

Expression vectors contain cis-acting regulatory regions that are operably linked in the vector to the receptor polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the receptor polynucleotides from the vector. Alternatively, a trans-acting factor may be supplied by the host cell. Finally, a trans-acting factor can be produced from the vector itself.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats. In a preferred embodiment, the promoter sequence is functional in kidney cells, such as the CMV promoter.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a receptor polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, adenso-associated virus, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The receptor polynucleotides, e.g., GPR54, can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS, HEK 293, and CHO cells, kidney cells, e.g., renal tubule cells, proximal tubule cells, glomeruli cells, and plant cells. It is understood that any of the established cell lines or any cell capable of being established are useful for producing long-term recombinant expression of the receptor polynucleotides. However, in some embodiments, it is useful to obtain recombinant expression in non-established cells as well.

As described herein, it may be desirable to express the polypeptide as a fusion protein. Accordingly, the invention provides fusion vectors that allow for the production of the receptor polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al., (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., (1988) Gene 69:301-315) and pET 11d (Studier et al., (1990) Gene Expression Technology: Methods in Enzymology 185:60-89).

Recombinant protein expression can be maximized in a host bacteria by providing a genetic background wherein the host cell has an impaired capacity to proteolytically cleave the recombinant protein. (Gottesman, Gene Expression Technology: Methods in Enzymology 185, Academic Press, San Diego, Calif. (1990) 119-128). Alternatively, the sequence of the polynucleotide of interest can be altered to provide preferential codon usage for a specific host cell, for example E. coli. (Wada et al., (1992) Nucleic Acids Res. 20:2111-2118).

The receptor polynucleotides can also be expressed by expression vectors that are operative in yeast. Examples of vectors for expression in yeast e.g., S. cerevisiae include pYepSec1 (Baldari, et al., (1987) EMBO J. 6:229-234), pMFa (Kurjan et al., (1982) Cell 30:933-943), pJRY88 (Schultz et al., (1987) Gene 54:113-123), and pYES2 (Invitrogen Corporation, San Diego, Calif.).

The receptor polynucleotides can also be expressed in insect cells using, for example, baculovirus expression vectors. Baculovirus vectors available for expression of proteins in cultured insect cells (e.g., Sf9 cells) include the pAc series (Smith et al., (1983) Mol. Cell Biol. 3:2156-2165) and the pVL series (Lucklow et al., (1989) Virology 170:31-39).

In certain embodiments of the invention, the polynucleotides described herein are expressed in mammalian cells using mammalian expression vectors. Examples of mammalian expression vectors include, but are not limited to, pCDM8 (Seed, (1987) Nature 329:840) and pMT2PC (Kaufman et al., (1987) EMBO J. 6:187-195).

The expression vectors listed herein are provided by way of example only of the well-known vectors available to those of ordinary skill in the art that would be useful to express the receptor polynucleotides. The person of ordinary skill in the art would be aware of other vectors suitable for maintenance propagation or expression of the polynucleotides described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

Mammalian cells include cellular test systems for ascertaining the effects of a given mutation on cell function, the function of a given mutation, the testing of compounds for the effect on a cell containing a given mutation, creating recombinant cells ex vivo for introduction into a subject, such as a transgenic animal or affected patient. In one embodiment, cells are derived from subjects or tissues known to be involved in kidney diseases or in the development of kidney diseases, such as kidney cancer, tumor and metastasis. Cells include, but are not limited to, kidney cells, renal cells, tubular cells, glomeruli cells. In addition, cells appropriate for test systems can include established cell lines, such as COS, HEK-293 kidney cells, and CHO cells.

The recombinant host cells can be prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the receptor polynucleotides can be introduced either alone or with other polynucleotides that are not related to the receptor polynucleotides such as those providing trans-acting factors for expression vectors. When more than one vector is introduced into a cell, the vectors can be introduced independently, co-introduced or joined to the receptor polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulafion of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the receptor polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Host cells expressing the nucleic acids and polypeptides described herein, particularly recombinant host cells, have a variety of uses in the context kidney diseases, such as kidney cancer, tumors, metastasis, ovarian cancer, endometrial cancer, and breast cancer. Vectors and host cells provide cellular test systems for ascertaining the effect of a test compound, e.g., an agonist, on a kidney host cell expressing a GPR54 protein. The GRP54 can be introduced into a given kidney host cell, e.g., HEK-293 kidney cells and assays are performed for cellular changes including biochemical, morphological, functional, gene expression, cellular changes, and the like.

In a further embodiment, a Kiss1 gene encoding metastin protein can be introduced into a given kidney host cell, e.g., HEK-293 kidney cells and assays are performed for cellular changes including biochemical, morphological, functional, gene expression, cellular changes, and the like. In yet a further embodiment, a Kiss1 gene encoding metastin protein can be introduced into a cell ex vivo so that the cell can be introduced back into its host, such as a transgenic animal or affected subject. Preferred cells are derived from a subject with kidney cancer or tumors or the risk of developing the kidney cancer or tumors, or from tissues and cells known to be involved in kidney cancer or tumors. Cells include, but are not limited to, renal cells, kidney cells, renal cells, tubular cells, and glomeruli cells.

The host cells expressing the polypeptides described herein, and particularly recombinant host cells, have a variety of uses. First, the cells are useful for producing receptor proteins or polypeptides that can be further purified to produce desired amounts of receptor protein or fragments. Thus, host cells containing expression vectors are useful for polypeptide production.

Host cells are also useful for conducting cell-based assays involving the receptor or receptor fragments. Thus, a recombinant host cell expressing a native receptor is useful to assay for compounds that stimulate or inhibit receptor function. This includes ligand binding, gene expression at the level of transcription or translation, G-protein interaction, and components of the signal transduction pathway.

Host cells are also useful for identifying receptor mutants in which these functions are affected. If the mutants naturally occur and give rise to a pathology, host cells containing the mutations are useful to assay compounds that have a desired effect on the mutant receptor (for example, stimulating or inhibiting function) which may not be indicated by their effect on the native receptor.

IX. Functional Assays

In one aspect, the invention pertains to screening for agents that modulate the activity of the receptor, e.g., GPR54. An agent which binds a GPR54 or a functional variant thereof can be studied in one or more suitable assays to determine if said agent can modulate (inhibit (reduce or prevent) or promote) one or more functions of GPR54. For example, an agent can be tested in an extracellular acidification assay, calcium flux assay, ligand binding assay, chemotaxis assay or assay which monitors degranulation or inflammatory mediator release (See e.g., Hesselgesser et al., (1998) J. Biol. Chem. 273(25):15687-15692 and WO 98/02151).

For example, an agent which binds to a GPR54 can be tested in suitable cells, for example, cell lines, recombinant cells or isolated cells which express a GPR54 or have been engineered to express GPR54. Suitable cell lines include, but are not limited to, Chinese Hamster Ovary cells (CHO), melanoma cells, HeLa cells, MDCK cells, HEK293 cells, and W138 cells, melanoma-derived LOX cell line as well as breast (MDA435) or colon (SW480, HT29, LoVo) cell lines which are available from the American Type Culture Collection (ATCC, Bethesda Md.).

An agent which binds to a GPR54 can also be assessed by monitoring cellular responses induced by active receptor, using suitable cells which express a GPR54 or a functional variant thereof. For instance, endocytosis, GPCR-internalization can be monitored by methods known in the art (See e.g., Norman et al., (2000) FEBS Lett 484:179-183; Kanzaki (2000) J. Biol. Chem. 275:7167-7175; Haugh et al., (1999) J Biol Chem 274:8958-8965; Seastone et al. (1999) Mol Biol Cell 10:393-406; and Benten et al., (1999) Mol Biol Cell 10:3113-3123).

In another embodiment, cells expressing a GPR54 or a functional variant thereof are combined with a ligand of GPR54 (e.g., metastin), an agent (e.g., FITC) to be tested can be added before, after, or simultaneous therewith, and Ca²⁺ flux can be assessed. The calsium flux can be used to demonstrate that there activity is not perturbed by the agent, and that the ligand (e.g., metastin) binds to the receptor and continues to target the receptor. In this embodiment, the entire complex of the agent-ligand may continue to target the rector and may be internalized.

The binding assays and functional assays described above can be used, alone or in combination with each other or other suitable methods, to detect or identify agents which bind a GPR54 protein and/or modulators (inhibitors, promoters) of a GPR54 protein function. The in vitro methods of the present invention can be adapted for high-throughput screening in which large numbers of samples are processed (e.g., a 96-well format). Cells expressing a GPR54 or a functional variant thereof at levels suitable for high-throughput screening can be used, and thus, are particularly valuable in the identification and/or isolation of agents which bind GPR54, and modulators of GPR54 function. Expression of GPR54 can be monitored in a variety of ways. For instance, expression can be monitored using antibodies of the present invention which bind receptor or a portion thereof. Also, commercially available antibodies can be used to detect expression of an antigen- or epitope-tagged fusion protein comprising a receptor protein or polypeptide (e.g., FLAG tagged receptors), and cells expressing the GPR54 at the desired level can be selected (e.g., by flow cytometry).

Assays involving the ligand, metastin, as a control can be used. Metastin is commercially available from Peptides International Louisville, or Phoenix Pharmaceuticals, Inc., Belmont, Calif. Other test agents can be added to determine if they behave in a similar fashion to metastin. Peptides that are analogous to metastin can be chemically synthesized tested. Modified versions of peptides of metastin can be chemically synthesized and tested. These modifications include those that result in a higher potency of interaction of the ligand with the GPR54 protein.

X. Diagnostic Applications

In one aspect, the invention pertains to diagnostic screening assays used to detect the presence of cancers involving tubular organs, such as kidney cancer, monitor the progression of the diseases and monitor the effect of treatment and therapies on the diseases. In one embodiment, antibodies of GPR54 can be used to detect the presence of GPR54 on the surface of cells. The antibodies can be used to detect and/or quantify cells expressing a GPR54. In one embodiment, the antibodies can be used to sort cells which express GPR54 from among a mixture of cells. Suitable methods for counting and/or sorting cells can be used for this purpose (e.g., flow cytometry, fluorescence activated cell sorting (FACS)). Cell counts can be used in the diagnosis of diseases or conditions in which an increase or decrease in cell types (e.g., kidney cells) is observed.

Furthermore, the antibodies can be used to detect or measure expression of GPR54. For example, antibodies can be used to detect or measure a GPR54 in a biological sample (e.g., cells, e.g., kidney cells, tissues, biopsy specimens, or body fluids such as blood, serum, plasma, saliva). Suitable assays can be used to assess the presence or amount of GPR54 protein. These assays include, but are not limited to, immunological and immunochemical methods such as flow cytometry (e.g., FACS analysis) and enzyme-linked immunosorbent assays (ELISA), including chemiluminescence assays, radioimmunoassay, immuno-blot (e.g., western blot) and immunohistology. Generally, a sample and antibody of the present invention are combined under conditions suitable for the formation of an antibody-GPR54 complex, and the formation of antibody-receptor complex is assessed (directly or indirectly).

The level of expression of a GPR54 protein or variant can also be used to correlate increased or decreased expression of a GPR54 protein with a particular disease or condition, and in the diagnosis of a disease or condition in which increased or decreased expression of a mammalian GPR54 protein occurs (e.g., increased or decreased relative to a suitable control, such as the level of expression in a normal individual). For example, the presence of an increased level of GPR54 reactivity in a kidney sample obtained from an individual is indicative of kidney cancer or tumor. The level of expression of GPR54 on the surface of cells can also be correlated with disease susceptibility, progression of disease or risk of developing the disease.

Similarly, the course of therapy can be monitored by assessing GPR54 immunoreactivity in a sample from a subject. For example, antibodies against GPR54 can be used to monitor the number of cells expressing GPR54 in a sample (e.g., blood, tissue) from a subject being treated with an anti-cancer agent.

Methods of Therapy

Modulation of mammalian GPR54 function according to the present invention, through the inhibition or promotion of at least one function characteristic of a mammalian GPR54 protein, provides an effective and selective way of inhibiting or promoting receptor-mediated functions. Thus, agents which can modulate GPR54 function, including ligands, inhibitors and/or promoters, such as those identified as described herein, can be used to modulate cell function, cell proliferation, cell inhibition, cell growth, motility and the like).

In one aspect, the present invention provides a method of modulating (e.g., inhibiting cell function) a response associated with GPR54 administering an effective amount of an agent which inhibits GPR54 function to the subject. In one embodiment, an effective amount of an agent which inhibits one or more functions of a GPR54 protein (e.g., a human GPR54) is the administration of anti-GPR54 antibodies. As a result, one or more processes involving GPR54, such as cell function, e.g., cell proliferation, chemotaxis, exocytosis, can be inhibited.

Thus, the invention relates to a method of treating a subject having an a disease or disorder associated with GPR54, comprising administering an effective amount of an antagonist of GPR54. In a particular embodiment, the subject has a cancer associated with elevated levels of GPR54. The cancer can be one associated with a particular organ, such as the liver. The elevated levels of GPR54 are those that are higher than an organ with normal function that is not afflicted with cancer.

XI. Pharmaceutical Compositions

The receptor nucleic acid molecules, protein (particularly fragments such as the amino terminal extracellular domain), modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a receptor protein or anti-receptor antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (See e.g., Chen et al., (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

Other embodiments and used of the invention will be apparent to those skilled in the art from consideration of the specification and practice of the invention disclosed herein. All U.S. Patents and other references noted herein for whatever reason are specifically incorporated by reference.

### EXAMPLES

### Example 1

### Extraction of RNA

Total cellular RNA was extracted from snap frozen tissue samples using standard protocols. cDNA was synthesized from 10 ug of total RNA using the Superscript Kit (BRL) with modifications described in detail previously (Byrne, M. C., M. Z. Whitley, and M. T. Follettie. 2000. Preparation of mRNA for expression monitoring. In: Current Protocols in Molecular Biology. John Wiley and Sons, Inc. New York). First strand synthesis was carried out at 50° C. to prevent mispriming from ribosomal RNA and utilized a T7 RNA polymerase promoter containing poly-T primer (T7T24) for subsequent in vitro antisense RNA (cRNA) amplification and biotin labeling. cDNA was purified using BioMag Carboxyterminated beads (Polysciences) according to manufactures instructions, and eluted in 48 ul of 10 mM NaAcetate pH7.8.

### Example 2

### In vitro Transcription

In vitro T7 polymerase driven transcription reactions for synthesis and biotin labeling of antisense cRNA was performed. The cRNA was purified using Qiagen RNeasy spin column purification and cRNA fragmentation was carried out as described in (Byrne et al. ibid). GeneChip® hybridization mixtures contained 10 ug fragmented cRNA, 0.5 mg/ml acetylated BSA, 0.1 mg/ml herring sperm DNA, in 1×MES buffer in a total volume of 200 μl as per manufacture's instructions. Reaction mixtures were hybridized for 18 hr at 45° C. to Affymetrix human Hg_U95 oligonucleotide arrays. The hybridization mixtures were removed and the arrays were washed and stained with Streptavidin R-phycoerythrin (Molecular Probes) using the GeneChip® Fluidics Station 400. The arrays were scanned with a Hewlett Packard GeneArray Scanner following manufacture's instructions. Fluorescent data was collected and converted to gene specific difference averages using MicroArray Suite 4.0 software.

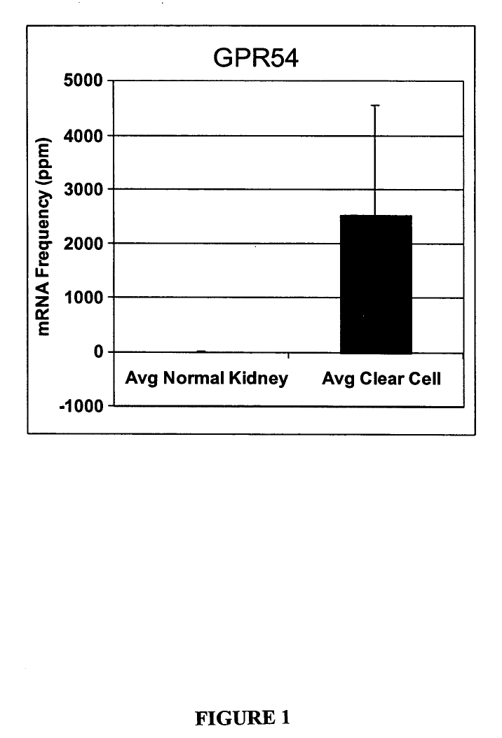

The results from these experiments are shown in Table 1 and FIG. 1. GPR54 fluorescence difference average in normal kidney samples averaged −1.8±11.7, and was absent (i.e., zero) in all 28 normal patient samples. In contrast, the fluorescence difference average in Clear kidney cancer samples averaged 2514±2040, and was called “Present” in 29 of 30 patient samples with Clear Cell Carcinoma (i.e. present in 97% of patients with Clear Cell Carcinoma). The data clearly shows the elevated levels of GPR54 in patients with Clear Cell Carcinoma.

TABLE 1
Increased Expression of GRP54 in kidney Cancer Cells

| | Normal Kidney Tissue | Clear Cell Carcinoma |
|---|---|---|
| | n = 28 | n = 30 |
| Average Expression¹ | −1.8 ± 11.7 | 2514 ± 2040 |
| Samples Called Present² | 0 present/28 patients | 29 present/30 patients |
| Range | −29.1 to 17.3 | −6.1 to 7274³ |
| Average Fold Change | | 503-fold |
| Student t-Test Statistical | | P = 2 × 10⁻⁷ |
| Analysis | | |

¹Expression measured as fluorescence intensity difference between perfect and single mismatched nucleotide probe averaged over gene-specific probes set and is directly related to mRNA concentration.
²In addition to fluorescence intensity measurement of concentration, software algorithms make an absolute call of mRNA presence or absence based on the fraction of positive and negative responding oligo-probe pairs.
³GPR54 mRNA was called absent in a single Clear Cell kidney cancer sample with measured −6.1 average fluorescence difference. Of the 29 samples called present, expression ranged from 66.9 to 7274.0.

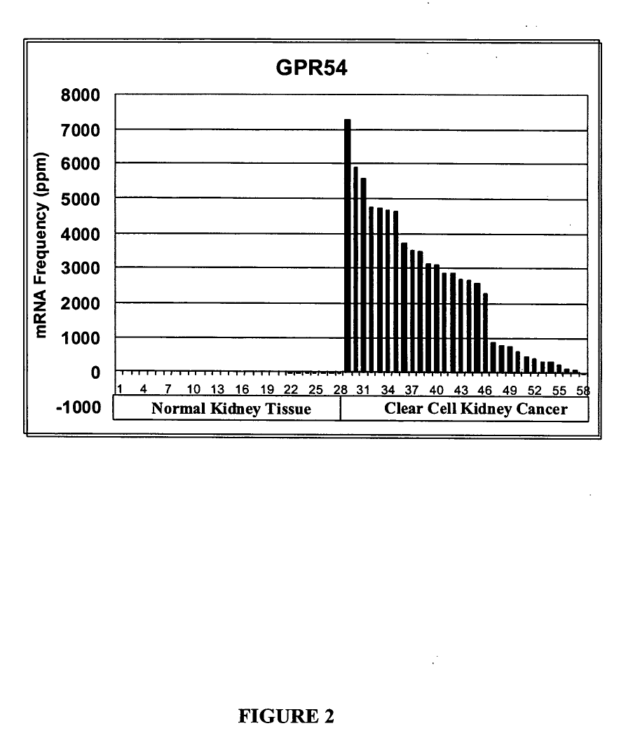

FIG. 2 is a bar graph showing GPR54 mRNA expression in individual normal kidney and clear cell cancer samples. The mRNA expression of both normal and kidney cancer samples were separately sorted to illustrate the range in expression measured. The graph illustrates that the majority of patients with Clear Cell Carcinoma express elevated levels of GPR54 to varying degrees, ranging from over 7000 ppm to about 500 ppm. A small minority of patients with Clear Cell Carcinoma (approximately 3%) express GPR54 at very low levels. In contrast, sampled from normal kidney tissue do not express GPR54. Thus, the GPR54 marker showed a differential expression with a statistical difference between diseased and normal tissue. The GPR54 is elevated in diseased cells.

### Example 3

### Vector Construction for GPR-54 Expression in Cells

This examples describes how to generate a construct having the human GPR54 gene for expression in cells, e.g., mammalian cells. The cDNA clone having complete coding sequence for human GPR54 protein was obtained. The DNA sequence for the entire coding region (1197 base pairs) was confirmed by nucleotide sequence analysis for both the coding (5′ to 3′) and opposite (3′ to 5′) strands. The sequence analysis confirmed the clone as being a full length DNA source for expressing human GPR54 mRNA.

The cDNA encoding cloned GPR54 was cloned into the mammalian expression vector pSPORT6 that contains the CMV promoter for mRNA expression in mammalian cells. The gene can be introduced into mammalian cell lines using standard lipid-based transfection protocols (e.g., Lipofectamine 2000; In Vitrogen). Increased expression of the human GPR54 can be measured by measuring GPR54-specific mRNA levels (Northern or PCR analysis) or by measuring protein expression with commercially available anti-GPR54 specific antibodies (LifeSpan).

### Example 4

### Delivery of Calicheamicin Into Cells Via GPR54

This example describes how to specifically deliver and internalize toxins, such as calicheamicin into cells overexpressing GPR54. The gene encoding human GPR54 can be cloned into a mammalian expression vector under the control of a strong transcriptional promoter (e.g., CMV promoter), as described above. The mammalian expression vector can be transfected into cancer cell lines and stable lines selected with vector encoded G418 resistance. Expression of the GPR54 mRNA can be confirmed by TaqMAN or Northern analysis and protein confirmed by Western analysis with antibodies purchased from LifeSpan Inc.

GPR54 encoding cDNA transfected mammalian cultures overexpressing human GPR54 can also be used to measure differential uptake or internalization of fluorescent-labeled peptide fragments from the Kiss-1 gene product (metastatin) relative to vector-alone control transfections. Metastin or peptide fragments thereof can be synthesized with a covalently attached amino-terminal fluorescein isothiocyanate (FITC) tag. GPR54 receptor mediated endocytosis can be monitored by fluorescence microscopy and demonstrated by internalization of FITC-ligand in GPR54 expressing cell lines, using vector-alone transfected cell lines as control. Additional controls include inhibition of fluorescent signal internalization with unlabeled peptide ligand(s) as opposed to randomized peptides of identical amino acid composition.

The internalization of FITC-tagged ligands by GPR54 mediated endocytosis can be demonstrated. The metastin-sequence derived synthetic ligands can be covalently attached to N-acetyl-gamma calicheamicin dimethyl hydrazide via bifunctional AcBut (4-[4′-acetylphenoxyl] butanoic acid linker. The ligand-toxin reagent can be used to demonstrate selective killing of cancer derived cells expressing GPR54 protein relative to vector-alone transfected controls. Cell death can be monitored by standard proliferation assays including measuring incorporation of radiolabeled nucleotides into DNA as function of time or by calorimetric assay of reduction of the tetrazolium salt MTT as assay of mitochondrial function. Specificity of drug delivery via GPR54 of the ligand peptide-toxin construct can be shown by demonstration of the requirement for GPR54 expression in target cell and metastin-derived sequence specificity in ligand-toxin conjugate for maximal drug-induced toxicity. Similarly, specificity of targeting can be demonstrated by competition with metastin sequence derived peptide ligand(s) alone that block specific uptake of ligand-toxin conjugates.

Additionally, the GPR54 transfected mammalian cultures can be used to demonstrate increased anti-proliferative activity of peptide coupled toxins such as calicheamicin relative to equimolar concentrations of calicheamicin alone.

### Example 5

### Profiling Downstream Genes Resulting from Metastin/GPR54 Activation

Downstream genes resulting from metastin/GPR54 activation can be assessed by detecting the alteration of gene expression profile of these genes mediated by stimulation GPR54 with ligand. The downsteam genes required for cancer cell metastasis can be identified by the alteration of gene expression profile mediated by stimulation GPR54 with a ligand.

This can be accomplished by using cancer cell lines transfected with the GPR54 receptor gene expression construct. The cell lines will be stimulated with a ligand agonist (e.g., metastin or metastin derived peptide ligands). The mRNA can be isolated from the stimulated cells and unstimulated control cells, and the cellular mRNA concentration can be profiled using Affymetrix oligonucleotide arrays to detect alterations in down stream genes required for cancer cell metastasis. Suitable cell lines include the melanoma-derived LOX cell line as well as breast (MDA-435) or colon (SW480, HT29, LoVo) cell lines (Available from American Type Culture Collection (Rockville, Md.)). The ligand/GPR54 mediated gene expression can be measured from cells grown either in-vitro or in mouse xenograft models. The mRNA expression altered by ligand stimulation of GPR54 may identify key genes in the metastatic phenotype of cancers and identify novel targets for chemotherapeutic intervention.

1. A method for identifying compounds that suppress G-protein coupled receptor-54 (GPR54) expression on cells, comprising:
providing a cell that expresses GPR54 on its cell surface;
measuring the expression level of the GPR54 receptor on a cell surface in the presence of a test compound; and
comparing the measured expression level with a predetermined value whereby useful compounds can be identified when the expression level of the G-protein coupled receptor in the presence of the test compound is lower than the predetermined value.

2. The method of claim 1, wherein the cell expresses heterologous GPR54.

3. The method of claim 1, wherein the expression level is determined by measuring the mRNA levels of GPR54.

4. The method of claim 1, wherein the expression level is determined by measuring the protein level of GPR54.

5. The method of claim 1, wherein the compound is an agonist of GPR54.

6. The method of claim 5, further comprising the step of measuring a signal transduction activity induced by the agonist.

7. The method of claim 6, wherein the step of measuring the signal transduction activity comprises measuring levels of at least one protein involved in a G-protein phospholipase C (PLC) pathway.

8. The method of claim 6, wherein the step of measuring the signal transduction activity involves measuring the interaction of the GPR54 with at least one arrestin protein.

9. A method of assessing a cell proliferation disease comprising:
measuring the expression level of a G-protein coupled receptor-54 (GRP54) in a sample from a subject; and
comparing the expression levels of GRP54 with a predetermined value, whereby a significantly higher expression level is an indicator of a disease state.

10. The method of claim 9, wherein the sample comprises cells collected from the kidney.

11. The method of claim 9, wherein the test sample comprises cells collected from blood.

12. The method of claim 9, wherein the level of expression of the GPR54 in the test sample differs from the normal level of expression of GPR54 in a subject not afflicted with kidney cancer by a factor of at least about 2.

13. The method of claim 9, wherein the level of expression of the GPR54 in the test sample differs from the normal level of expression of GPR54 in a subject not afflicted with kidney cancer by a factor of at least about 3.

14. The method of claim 9, wherein the cell proliferation disease is kidney cancer.

15. The method of claim 14, further comprising exposing the kidney cancer cell to a therapeutically effective amount of a GPR54 binding agent, and measuring the expression level of the GPR54 receptor, wherein a reduction in the expression level of the GPR54 receptor indicates that the cell has been specifically targeted.

16. The method of claim 15, wherein the binding agent is a GPR54 agonist.

17. The method of claim 16, wherein the agonist is metastin or a fragment thereof.

18. The method of claim 15, wherein the binding agent is an anti-GPR54 antibody.

19. The method of claim 15, wherein the binding agent further comprises a toxin.

20. The method of claim 19, wherein the toxin is calicheamicin.

21. The method of claim 19, wherein the toxin is selected from the group consisting of disulphide analogs of calicheamicin, dihydro derivatives of calicheamicin, N-acylated derivatives of calicheamicin, and esperamicins

22. The method of claim 15, wherein the binding agent interacts with the GPR54 and undergoes endocytosis.

23. The method of claim 15, wherein the binding agent interacts with the GPR54, such that the GPR54 from the cell surface is internalized and binds with at least one arrestin protein.

24. The method of claim 16, wherein the GPR54 agonist interacts with the GPR54 to activate a G-protein phospholipase C (PLC) pathway.

25. A method of inhibiting cell function in a subject expressing aberrant levels of G protein-coupled receptor-54 (GPR54) by exposing the subject to a therapeutically effective amount of a GPR54 binding agent.

26. The method of claim 25, wherein the binding agent is a GPR54 agonist.

27. The method of claim 26, wherein the agonist is metastin or a fragment thereof.

28. The method of claim 25, wherein the binding agent is an anti-GPR54 antibody.

29. The method of claim 25, wherein the binding agent further comprises a toxin.

30. The method of claim 29, wherein the toxin is calicheamicin.

31. The method of claim 29, wherein the toxin is selected from the group consisting of disulphide analogs of calicheamicin, dihydro derivatives of calicheamicin, N-acylated derivatives of calicheamicin, and esperamicins.

32. The method of claim 25, wherein the binding agent interacts with the GPR54 and undergoes endocytosis.

33. The method of claim 25, wherein the binding agent interacts with the GPR54, such that the GPR54 from the cell surface is internalized and binds with at least one arrestin protein.

34. The method of claim 26, wherein the GPR54 agonist interacts with the GPR54 to activate a G-protein phospholipase C (PLC) pathway.

35. A method of inhibiting cell function in a subject with kidney cancer comprising:
administering a vector comprising a KiSS-1 gene to kidney cells of the subject, wherein the KiSS-1 gene is operably linked to a promoter functional in kidney cells; and
expressing the KiSS-1 gene to produce metastin, such that the increased amount of metastin in the cell interacts with G-protein coupled receptor-54 (GPR54) to inhibit cell function.

36. The method of claim 35, wherein the step of expressing the KiSS-1 gene to produce metastin further results in inhibiting cell metastasis.

37. The method of claim 35, wherein the vector is selected from the group consisting of adeno-associated virus, lentivirus, adenovirus, rectrovirus.
